(19) United States
(12) Patent Application Publication
(10) Pub. No.: US 2026/0263200 A1
(43) Pub. Date: Sep. 10, 2026

(54) SMART LOCKING HYGIENIC TOOTHBRUSH AND ORAL HEALTH SYSTEM

(71) Applicants: Nayeli Saenz Monroy, National City, CA (US); Martin Saenz Zarate, National City, CA (US)

(72) Inventors: Nayeli Saenz Monroy, National City, CA (US); Martin Saenz Zarate, National City, CA (US)

(21) Appl. No.: 19/071,376

(22) Filed: Mar. 5, 2025

(51) Int. Cl. A61C 17/22; A61L 2/00

(52) U.S. Cl. CPC A61C 17/221; A61L 2/10; A61L 2103/05; A61L 2202/121

(57) ABSTRACT

A smart locking hygienic toothbrush and oral health system is disclosed, including a toothbrush including a handle portion and a head portion, the head portion including a bristle cluster. A sensor array is in communication with a housing of the toothbrush to detect the usage of the toothbrush and a lockable enclosure to protect usage and exposure of the toothbrush. A microprocessor is in electrical communication with the sensor array. One or more I/O devices are in electrical communication with the microprocessor, the I/O devices to provide an alert, a sound, or an indication. A protective case is dimensioned to at least partially receive the toothbrush and to encase the head portion.

### CROSS-REFERENCE TO RELATED APPLICATIONS

The present application claims priority to U.S. Provisional Application No. 63/533,023 filed Aug. 16, 2023, titled “Smart Locking Hygienic Toothbrush and Oral Health System,” which is hereby incorporated by reference in its entirety.

### TECHNICAL FIELD

The embodiments disclosed herein generally relate to oral care devices and more particularly to a smart toothbrush and oral health system.

### BACKGROUND

Oral hygiene is the practice of keeping the oral cavity clean to prevent diseases and various other health problems by regularly brushing and cleaning one's teeth, gums and tongue. It is important that hygienic practices are carried out on a regular basis to avoid tooth decay and gum disease as well as to maintain the whiteness of the teeth. It is recommended that individuals develop a regimen, such as brushing the teeth in the morning, after meals, and/or before bedtime. Maintaining an adequate regimen can provide difficult for many. Epidemiology shows that failure to following a suitable regimen reduces the person's ability to stave off bacteria that is proving to be more and more impervious to antibiotics and leads to many unrecognized and unconsidered health problems.

Many products exist which aid maintaining dental hygiene. In particular, the toothbrush is perhaps the most widely used oral hygiene tool and consists of a handle and head comprised of tightly clustered bristles. The user applies toothpaste to the top of the bristles before brushing the teeth, gums, and tongue. Various forms exist including the manual toothbrush which is operated by the user manually moving the bristles throughout the oral cavity, while electric toothbrushes offer automated multi-directional movement of the bristles.

It is commonly known that toothbrushes harbor potentially harmful microorganisms and that toothbrush contamination presents a role in disease transmission and may increase the risk of an oral infection as they serve as a reservoir for microorganisms. Contamination of the toothbrush occurs soon after initial use and increases with repeated use. They may become contaminated from the oral cavity, the environment, the hands, from aerosols and from storage containers. Moreover, unauthorized third party users commonly known as toothbrush thieves which could be anyone including, spouses, paramours, siblings, friends, hunkies or dormmates as unwanted interlopers presenting additional risk to the potentiality of contagion of all sorts foreign to and hazardous to the user's biology; physical and mental well-being often simply irksome and infuriating to the rightful owner of the toothbrush.

Diseases from poor oral hygiene include Cardiovascular disease, Alzheimer's disease, diabetes, Pregnancy delivery issues, Obstructive sleep apnea, Respiratory problems, Cancer, Rheumatoid arthritis, Osteoporosis, Kidney disease, Prostate/erectile dysfunction, Periodontal disease, Obesity, COVID-19, and Infertility.

While electric toothbrushes have become increasingly commonplace, there are no self securing, self-monitoring, self-cleaning connected or autonomous electronic toothbrushes or independent toothbrush guards or bases which are available sharing these qualities

### SUMMARY OF THE INVENTION

This summary is provided to introduce a variety of concepts in a simplified form that is further disclosed in the detailed description of the embodiments. This summary is not intended for determining the scope of the claimed subject matter.

An electronic toothbrush system is disclosed, including a toothbrush including a handle portion and a head portion, the head portion including a bristle cluster. A sensor array is in communication with a housing of the toothbrush to detect the usage of the toothbrush. A microprocessor is in electrical communication with the sensor array. One or more I/O devices are in electrical communication with the microprocessor, the I/O devices to provide an alert, a sound, or an indication. A protective case is dimensioned to at least partially receive the toothbrush and to encase the head portion.

In one aspect, the sensor array is comprised of a motion sensor to detect motion of the toothbrush.

In one aspect, the sensor array includes a kinetic motion detector.

In one aspect, the sensor array includes a biometric sensor(s) capable of scanning a biometric and biofeedback element(s) of a human.

In one aspect, the system includes a locking mechanism to selectively secure the protective case in a closed position. The locking mechanism may also function as a preventing unauthorized use of the electronic toothbrush system. This is performed using the various biometric sensors which aid in the identification of the user and their operation of the locking mechanism once the user has been properly identified. If an unauthorized user is detected, the device will remain locked within the locking protective case or enclosures.

In some embodiments, the locking mechanism is a magnetic or mechanical locking mechanism prevent the protective case from opening. The locking mechanism is a true-locking mechanism which prevents any unwanted or unpermitted access to the toothbrush.

In one aspect, one or more UV lights provide an antiseptic environment within an interior of the protective case for posterity while at the same time providing the antiseptic environment for the brushing area within the user's oral cavity, wherein the one or more UV lights are oriented to emit UV light towards the bristle cluster of the toothbrush.

In one aspect, the one or more UV lights are mounted on the head of the toothbrush.

In one aspect, the one or more UV lights are mounted on an interior surface of the protective case.

In one aspect, the toothbrush is mountable to a base to secure the toothbrush while the toothbrush is inoperative, wherein the base includes a second sensor array including a at least one motion sensor and second biometrics sensor, wherein the base includes a second set of at least one I/O devices to provide a sound, an alert, or an indicator, and wherein the base includes a second microprocessor in operable communication with the second sensor array, the second set of at least one I/O devices, and the second biometrics sensor.

In one aspect, the electronic toothbrush system includes a computing system in operable communication with an application program operated by a user computing device. The application program is in communication, via a network or near-frequency communication protocol, to the toothbrush to execute computer-readable instructions to establish operational protocols to aid in the formation of a hygiene regimen carried out by the utilization of the toothbrush.

In one aspect, the application program provides a means of collecting and storing data within a database.

In one aspect, the application program is operable to transmit reminders and/or alerts to the user, parent, and/or caregiver to promote patient engagement.

In one aspect, the application program is in operable communication with one or more third-party platforms including telehealth platforms. The telehealth platforms may include dental telehealth platforms, orthodontist telehealth systems, and review of technique and hints and suggestions provided by the caregiver or a BOT via Artificial Intelligence learned over time of the characteristics of each individual patients care plan and their own specific needs or of concern.

In one aspect, the integration with telehealth and digital health platforms provides a means for scheduling appointments, via a scheduling module, with a dental, orthodontist, hygienist, and other healthcare professionals. The scheduling module may assist the user, parent, and/or caregiver in finding and procuring the services of healthcare professions, dentists, hygienists, orthodontists, etc. This process may be location-based, insurance-based, or otherwise determined by the user's preferences and needs.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. I illustrates a perspective view of the electronic toothbrush and protective case, according to some embodiments;

FIG. 2 illustrates a side elevation view of the electronic toothbrush and protective case, according to some embodiments;

FIG. 3 illustrates a perspective view of the electronic toothbrush and protective case, according to some embodiments;

FIG. 4 illustrates a perspective view of the base, according to some embodiments;

FIG. 5 illustrates a perspective view of the toothbrush and protective travel case, according to some embodiments;

FIG. 6 illustrates a block diagram of the computer system architecture, according to some embodiments;

FIG. 7 illustrates a block diagram of the application program and computing system, according to some embodiments;

FIG. 8 illustrates a block diagram of the computer system and databases used for the collection and storage of data, according to some embodiments;

FIG. 9 illustrates a block diagram of the electronic toothbrush system and associated electronic components of the system, according to some embodiments;

FIG. 10 illustrates a perspective view of the protective case 101 in a closed configuration, according to some embodiments;

FIG. 11 illustrates a perspective view of the protective case in an open configuration, according to some embodiments; and

FIG. 12 illustrates a perspective view of the base, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments described herein relate to an electronic toothbrush system which includes an electronic, manual toothbrush, flossing devices (eg., water picks or dental floss container), scrapers, tongue scrapers, or similar dental hygiene devices, a protective case, and in some instances a base. The system is in communication with a computer system operating an application program on a computing device (i.e., a computer, smartphone, tablet, etc.). The electronic toothbrush system includes one or more ultraviolet (UV) lights which emit UV light towards the elements of the toothbrush, including the bristle cluster, head portion, and/or the handle portion in order to sterilize, prevent or inhibit the growth of bacteria, yeast, and other contaminants on the toothbrush. The UV lights may also function to sterilize, prevent or inhibit the growth of bacteria, yeast, and other contaminants on or within the protective case.

The toothbrush, protective case, and/or the base are provided as separate components of the system. In such, the toothbrush can be separated and selectively stored within the protective case. The protective case provides a lockable housing which protects the toothbrush (or other oral hygiene device) as well as provides the cleaning, security, and other features described in the embodiments provided herein.

The electronic toothbrush system provides a replaceable, disposable, and/or biodegradable toothbrush which is cleaned by the UV lights provided on and/or within the protective case. It is known that a toothbrush should be changed or replaced about every 90 days, according to the American Dental Association. This is due to the exposure of the toothbrush to bacteria, yeast, and other contaminants, as well as the inherent wear-and-tear which damages the toothbrush, causing it to lose it efficacy. The embodiments provided herein mitigate this problem, at least in part, by providing a means of sanitizing or at least partially cleaning the toothbrush between uses. The protective case provides an enclosure which, through the use of UV light, provides the benefits described herein. In such, the electronic toothbrush system described herein provides an environmentally friendly solution to the aforementioned problems, as the disposal of toothbrushes has a significant impact on the environment.

It is to be understood, that while an electronic toothbrush is used by way of example, the dental device can include any type of electronic toothbrush, manually operated toothbrushes, flossing devices, water picks, scrapers, tongue scrapers, or similar dental hygiene devices. The accessories as described herein may each be operated in conjunction with, or separate from the base, locking protective case, and other electronic elements described herein.

In some embodiments, the components of the electronic toothbrush system (i.e., the toothbrush, the base, and the protective case) may be constructed of environmentally friendly materials known in the arts.

FIGS. 1-3 illustrates the toothbrush 100 and protective case 101. The toothbrush may be a manually operated toothbrush or an electronic toothbrush which is used as a component of the electronic toothbrush system described herein. The toothbrush 100 includes a bristle cluster 103 positioned on the head portion 105 of the toothbrush 100 and a handle portion 106 which allows the user to hold the toothbrush 100. The bristle cluster 103 includes a plurality of bristles 107 which extends substantially perpendicular to the front surface 109 of the head portion 105. The handle portion 106 may include a display, buttons, or other user interface which allows the user to control the functions of the toothbrush 100.

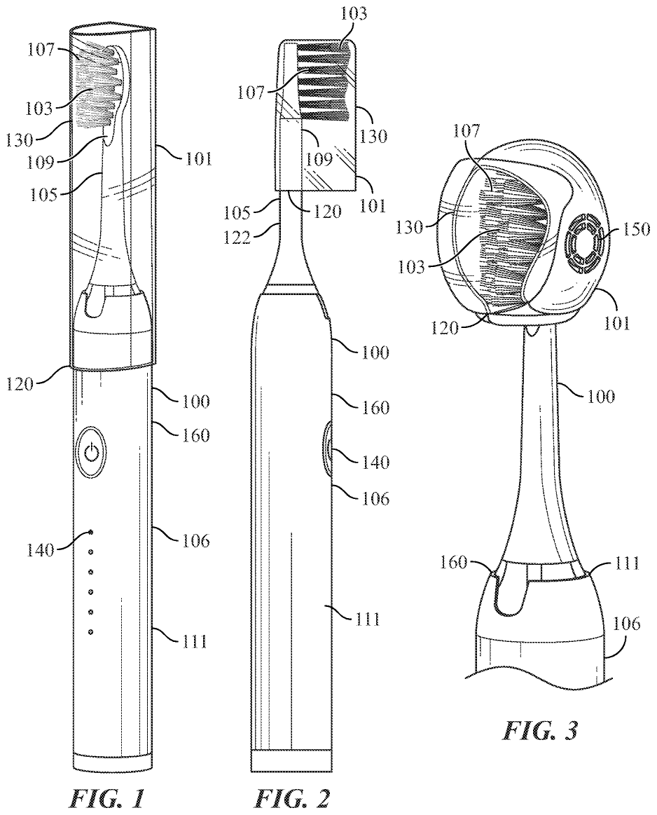

FIGS. 1-3 illustrates various examples of configurations of the protective case 101. FIG. 1 illustrates the protective case 101 having a configuration which encases the head portion 105 and at least a portion of the handle portion 106. The locking mechanism 120 is positioned to lockingly engage with the handle portion 106 when secured thereto. FIG. 2 illustrates the protective case 101 having a configuration which encases at least a portion of the head portion 105 and lockingly engaged thereto. The locking mechanism 120 may engage with the stem 122 of the head portion 105. FIG. 3 illustrates the protective case 101 having a configuration which encases a portion of the head portion 105 and is constructed having a spherical shape. In any example of the protective case 101, it is configured to house the bristle cluster 103 to prevent contamination.

In some embodiments, the locking mechanism 120 may function using a heat lock, electromagnetic lock, mechanical lock, or a friction-fit lock. The locking mechanism may be autonomously operated and/or manually operated using the I/O device(s) provided on the toothbrush or the application program. In an embodiment wherein the application program is used, the locking mechanism 120 is remotely operated by the user. The locking mechanism 120 performs the function of securing the protective case or travel case in a closed position.

In some embodiments, the locking mechanism 120 may also function as a preventing unauthorized use of the electronic toothbrush system. This is performed using the various biometric sensors which aid in the identification of the user and their operation of the locking mechanism once the user has been properly identified. If an unauthorized user is detected, the device will remain locked within the locking protective case.

In some embodiments, the locking mechanism 120 is in communication with the biometric sensor(s) to autonomously unlock the device in the presence of the user.

In some embodiments, the locking mechanism 120 may be operated by a numeric input provided by the user (e.g., a numeric passcode). The numeric passcode may be input electronically, or using tactile buttons positioned on any component of the system (e.g., the protective case, the base, or an external keypad). In another embodiment, the locking mechanism 120 and means for unlocking the device can be operated remotely using a smart device (such as the user interface of a smartphone). In another embodiment, the locking mechanism may be operated using the display.

In some embodiments, the biometric sensor(s) may include biofeedback sensor(s) which may detect and captured including various vital statistics in real time including a pulse rate, oxygen levels, temperature (whether ambient temperature, internal body temperature, skin temperature, etc.), and other biometrics known in the arts.

The protective case 101 includes one or more UV lights 130 which emit light in the UV spectrum in order to sterilize, prevent or inhibit the growth of bacteria, yeast, and other contaminants on the toothbrush or on or within the protective case. It should be understood that the UV lights may be positioned in an array on any component of the toothbrush 100.

In some embodiments, the UV light(s) may emit UV-A light in the UV light spectrum between 320-400 nm. UV-B light may also be emitted in the UV light spectrum between 280-320 nm. UV-C light may be emitted in the UV light spectrum between 200-280 nm. Each range within the UV light spectrum is known to have significant germicidal effects.

In some embodiments, the UV light(s) may be used as an oral hygiene element to direct UV light into the user's mouth while the user is performing an oral hygiene procedure (e.g., brushing their teeth, flossing, etc.).

In some embodiments, the UV light(s) function as a means of preserving the lifespan of the bristles of the toothbrush as well as the other elements of the oral hygiene device (i.e., preserving floss in the case where the oral hygiene device is a flossing device, or preserving elements of a water pick, etc.).

In some embodiments, the UV lights are in operable communication with the locking mechanism such that as the locking mechanism is activated, the UV lights are switched to an ON function and flash flush the environment with the effective use of the UV light via the light spectrum assignment. In another embodiment, the UV lights may be in communication with a timer, such that as the locking mechanism is activated the UV lights are switched to an ON function for a pre-programmed period of time.

In some embodiments, the protective case 101 includes one or more I/O devices 140 which may include various LED lights. The one or more I/O devices 140 may be used along with one or more speaker(s) and/or microphones 150 (see FIG. 3) to emit a sound and light indicator to indicate (via various colors of light and an audible sound) a status of the toothbrush, power storage device, network connectivity, etc.

In some embodiments, the LED lights and/or the speaker/or mic may be used to locate the toothbrush. For example, the speaker may emit an audible sound to help the user locate the brush. Separately or simultaneously, the LED lights may flash to aid in locating the toothbrush. One or more microphones may be included which can detect the user's voice, and in conjunction with the biometric sensor(s) be used to identify the user's voice to unlock the protective case. One skilled in the arts will readily understand that various additional use-cases may be implemented using the microphone. For example, the microphone may detect noises in the surrounding environment to detect the presence of the user.

In some embodiments, the one or more I/O devices 140 may include one or more buttons provided one the housing of the toothbrush 100. The buttons may be used as a user interface to control the various operational functions of the electronic toothbrush system.

In some embodiments, the toothbrush may include a QRL or linear bar code to provide a means of identifying and verifying the toothbrush. The QRL or linear bar code may be scanned by the user's computing device to identify the component it is printed on and associate the component with the user's account.

In some embodiments, a sensor array is provided within the housing 160 of the toothbrush and/or the protective case 101. The housing 160 may be defined as the entirety of, or a portion of, the handle portion 106 and the head portion 105. The housing 160 may be used as a term to describe each of the handle portion 106 and the head portion simultaneously.

In some embodiments, the handle portion 106 and the head portion 105 may releasably engage with one another to provide a two-piece assembly. This may be beneficial by allowing the head portion 105 to be replaced if the bristles 107 become damaged or worn. The two-piece assembly may also be beneficial when travelling, allowing the user to disconnect the head portion 105 from the handle portion 106, thus reducing storage space of the toothbrush 100.

In some embodiments, the handle portion I 06 utilizes ergonomic design to fit the bio feedback points in a human users' hands to align sensors to provide optimum feedback to the systems bio analysis capabilities via the physical device as data is collected frequently however selectively through the intelligent design of the physical form of the instrument in use of the system.

In some embodiments, the toothbrush 100 may be an electronic toothbrush 111 having an electric motor to provide oscillating, vibrating, gyrating, spinning or similar movement to the head portion 105 in order to move the bristle cluster in a manner which improves the cleaning efficiency and ability of the toothbrush. The electronic toothbrush 111 may include a power storage device or other means of storing and supply power to the electric motor.

In some embodiments, the toothbrush 100 may be a manually-operated toothbrush which requires the user to manually move the toothbrush 100 while brushing the teeth. It is to be understood that the embodiments may disclosed herein may be accomplished using either an electronic or manually-operated toothbrush. In the case that the toothbrush 100 is manually operated, the protective case 101 and/or base may be in communication with the computer system and application program, rather than the toothbrush 100 itself In the case wherein the toothbrush 101 is an electronic toothbrush, any combination of the toothbrush 100, protective case 101, and/or the base may be in communication with the computer system and application program.

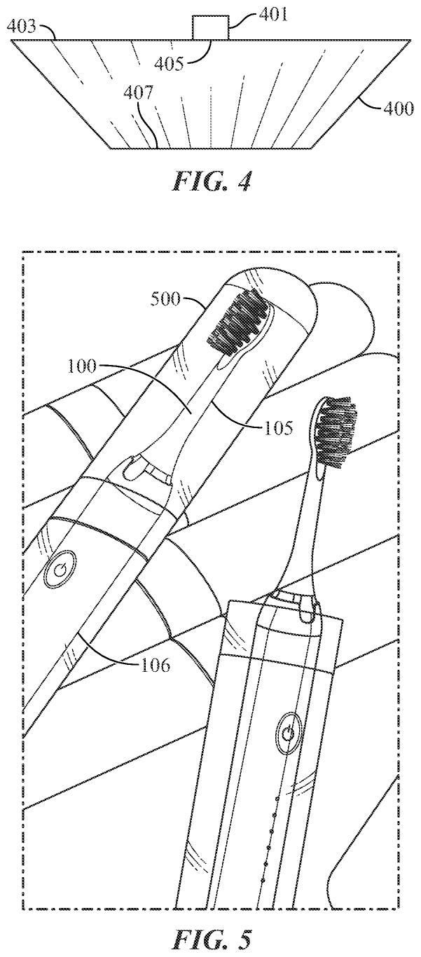

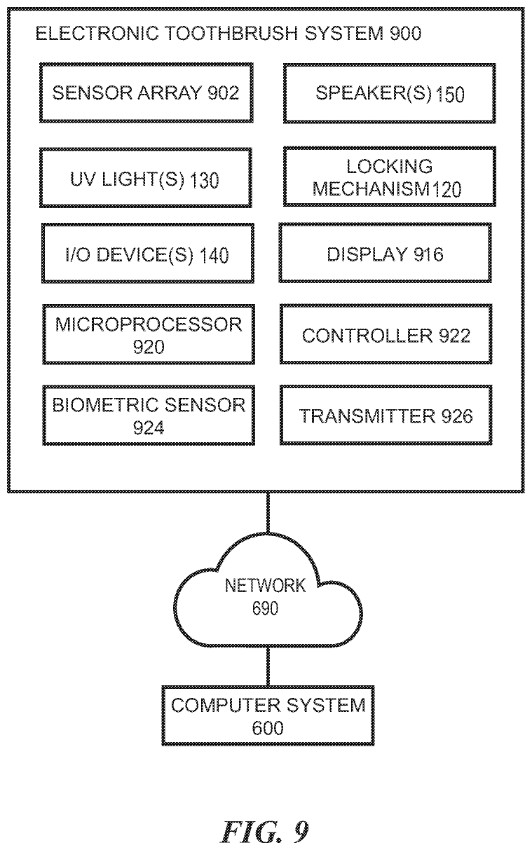

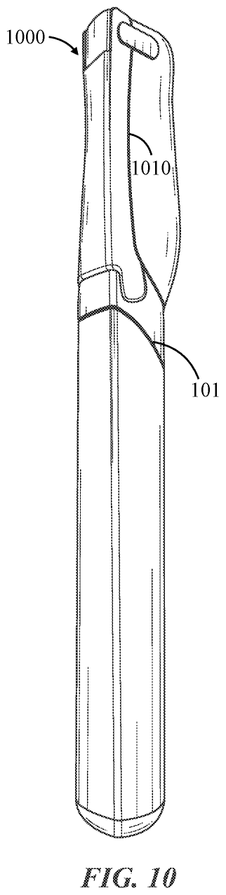

FIG. 4 illustrates a perspective view of the base 400. The base 400 includes a mounting element 401 which allows for the toothbrush to be secured to the base 400. The base 400 may be used as a means of conducting electricity to the toothbrush, acting as a charger. The base 400 may be in electrical connection to an electrical outlet which is used as a power source. Alternatively, the base 400 may include a power storage device to receive and store power, without being connected to an electrical outlet. The base 400 may include one or more elements of the sensor array, the biometric sensor(s), a memory, a transmitter, UV light(s), indicator, microprocessor, controller, display, locking mechanism, speaker(s), microphones, etc. as described in FIG. 9 and the disclosure provided herein.

In some embodiments, the power source can include rechargeable batteries, replaceable batteries, wireless charging devices, or may be wired to a wall outlet to provide power to the electrical components of the device.

In some embodiments, the base 400 includes a top surface 403 which includes the mounting element 401 whereon the toothbrush is placed. The mounting element 401 may include means of transmitting electrical power to the toothbrush to function as a charging port 405. The base 400 also includes a bottom surface 407 which provides a stable surface which can contact and be supported by a countertop, vanity top, or other surface.

In some embodiments, the base 400 is in communication, via network 690, to the computer system 600 to provide any functions described by the electronic toothbrush system 900 (see FIG. 9 and entirety of the disclosure provided herein).

FIG. 5 illustrates a perspective view of the toothbrush and protective travel case 500. The protective travel case may perform any functions described by the electronic toothbrush system 900 (see FIG. 9) and entirety of the disclosure provided herein. The protective travel case 500, may encase the entire toothbrush including the handle portion 106, head portion 105, bristle cluster 103, and other components of the toothbrush 100. The protective travel case 500 may be best suited for use during travel to protect the toothbrush from damage and contamination. The protective travel case 500 includes one or more UV lights to provide the on-demand sterilization of the toothbrush 100.

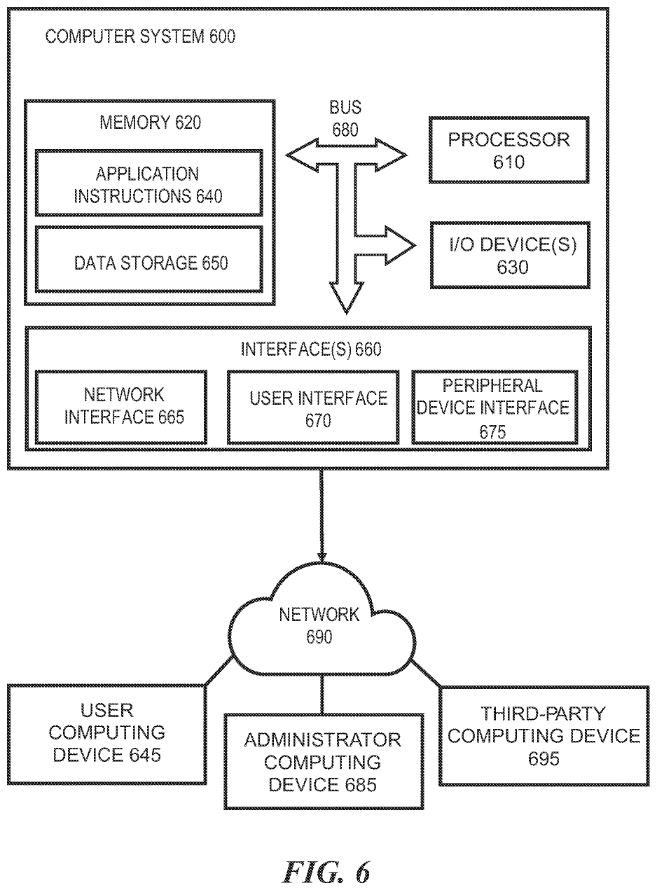

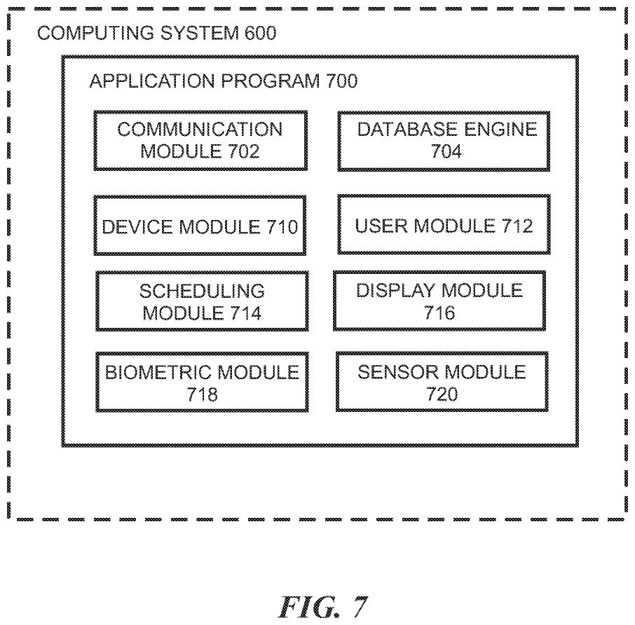

FIG. 6 illustrates an example of a computer system 600 that may be utilized to execute various procedures, including the processes described herein. The computer system 600 comprises a standalone computer or mobile computing device, a mainframe computer system, a workstation, a network computer, a desktop computer, a laptop, or the like. The computing device 600 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 600 includes one or more processors 610 coupled to a memory 620 through a system bus 680 that couples various system components, such as an input/output (I/O) devices 630, to the processors 610. The bus 680 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 600 includes one or more input/output (I/O) devices 630, such as video device(s) (e.g., a camera), audio device(s), and display(s) are in operable communication with the computer system 600. In some embodiments, similar I/O devices 630 may be separate from the computer system 600 and may interact with one or more nodes of the computer system 600 through a wired or wireless connection, such as over a network interface.

Processors 610 suitable for the execution of computer readable program instructions include both general and special purpose microprocessors and any one or more processors of any digital computing device. For example, each processor 610 may be a single processing unit or a number of processing units and may include single or multiple computing units or multiple processing cores. The processor(s) 610 can be implemented as one or more microprocessors, microcomputers, microcontrollers, digital signal processors, central processing units, state machines, logic circuitries, and/or any devices that manipulate signals based on operational instructions. For example, the processor(s) 610 may be one or more hardware processors and/or logic circuits of any suitable type specifically programmed or configured to execute the algorithms and processes described herein. The processor(s) 610 can be configured to fetch and execute computer readable program instructions stored in the computer-readable media, which can program the processor(s) 610 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum dot based transistors, switches, and gates, to optimize space usage or enhance performance of user equipment. A processor can also be implemented as a combination of computing processing units.

In some embodiments, the memory 620 includes computer-readable application instructions 650, configured to implement certain embodiments described herein, and a database 650, comprising various data accessible by the application instructions 640. In some embodiments, the application instructions 640 include software elements corresponding to one or more of the various embodiments described herein. For example, application instructions 640 may be implemented in various embodiments using any desired programming language, scripting language, or combination of programming and/or scripting languages (e.g., Android, C, C++, C #, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 640 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 610 such that the processor 610 can read information from, and write information to, the storage medium. In the alternative, the storage medium may be integrated into the processor 610. Further, in some embodiments, the processor 610 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 640 for carrying out operations of the present disclosure can be assembler instructions, instruction-set-architecture (ISA) instructions, machine instructions, machine dependent instructions, microcode, firmware instructions, state setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 640 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 640 can be downloaded to a computing/processing device from a computer readable storage medium, or to an external computer or external storage device via a network 690. A network adapter card or network interface in each computing/processing device receives computer readable program instructions from the network and forwards the computer readable application instructions 640 for storage in a computer readable storage medium within the respective computing/processing device.

In some embodiments, the computer system 600 includes one or more interfaces 660 that allow the computer system 600 to interact with other systems, devices, or computing environments. In some embodiments, the computer system 600 comprises a network interface 665 to communicate with a network 690. In some embodiments, the network interface 665 is configured to allow data to be exchanged between the computer system 600 and other devices attached to the network 690, such as other computer systems, or between nodes of the computer system 600. In various embodiments, the network interface 665 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 670 and the peripheral device interface 675.

In some embodiments, the network 690 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 690 can comprise copper transmission cables, optical transmission fibers, wireless transmission, routers, firewalls, switches, gateway computers and/or edge servers. The network 690 can represent a single network or multiple networks. In some embodiments, the network 690 used by the various devices of the computer system 600 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 600 may include a user computing device 645, an administrator computing device 685 and a third-party computing device 695 each in communication via the network 690. The user computing device 645 may be utilized a user (e.g., a healthcare provider) to interact with the various functionalities of the system including to perform patient rounds, handoff patient rounding responsibility, perform biometric verification tasks, and other associated tasks and functionalities of the system. The administrator computing device 685 is utilized by an administrative user to moderate content and to perform other administrative functions. The third-party computing device 695 may be utilized by third parties to receive communications from the user computing device, transmit communications to the user via the network, and otherwise interact with the various functionalities of the system.

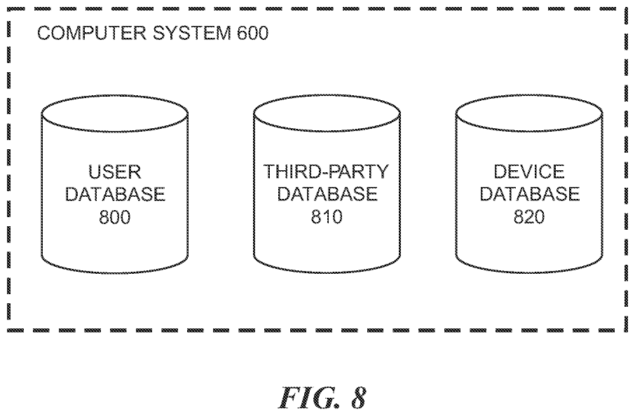

FIG. 7 illustrates an example computer architecture for the application program 700 operated via the computing system 600. The computer system 600 comprises several modules and engines configured to execute the functionalities of the application program 700, and a database engine 704 configured to facilitate how data is stored and managed in one or more databases (see FIG. 8). In particular, FIG. 7 is a block diagram showing the modules and engines needed to perform specific tasks within the application program 700.

Referring to FIG. 7, the computing system 600 operating the application program 700 comprises one or more modules having the necessary routines and data structures for performing specific tasks, and one or more engines configured to determine how the platform manages and manipulates data. In some embodiments, the application program 700 comprises one or more of a communication module 702, a database engine 704, a device module 710, a user module 712, a scheduling module 714, a display/projection module with stereoscopic capabilities 716, a biometric module 718, and a sensor module 720.

In some embodiments, the communication module 702 is configured for rece1vmg, processing, and transmitting a user command and/or one or more data streams. In such embodiments, the communication module 702 performs communication functions between various devices, including the user computing device 645, the administrator computing device 685, and a third-party computing device 695. In some embodiments, the communication module 702 is configured to allow one or more users of the system, including a third-party, to communicate with one another. In some embodiments, the communications module 702 is configured to maintain one or more communication sessions with one or more servers, the administrative computing device 685, and/or one or more third-party computing device(s) 695.

In some embodiments, the communication module 702 may transmit alerts to users of the system. Alerts may be related to notifications which correspond to events of a hygiene regimen. The events may include brushing one's teeth, cleaning the toothbrush, operating the UV lights to sterilize the toothbrush, scheduling and/or attending an appointment (e.g., a dental hygienist appoint, dentist appointment, orthodontist appointment, etc.). The alert and/or notification may be transmitted to the user computer device and/or the electronic toothbrush. For example, the alert sent to the toothbrush may emit a sound or light on the toothbrush, on the protective case, on the base, or other component. The alert may coincide with a notification transmitted to the user computing device.

In some embodiments, the communication module 702 is operable to transmit a signal to operate one or more speakers, microphones and/or one or more indicators on the electronic toothbrush, the base, and/or the protective case. The indicators may include lights which provide an indication of operational status (i.e., is the toothbrush on or off, is the cleaning sequence operating or completed, etc.). The indicators may be used to indicate a battery level of the base, the protective case, and/or the electronic toothbrush. The speakers may be used to transmit an audible alert to the user. Meanwhile, the microphone(s) may be used to receive audible or visual cues from the user and or instructor(s) including any proctor live, pre-recorded, or sentient in real time independent of source input re caregiver bot or program which instruct the system to perform various functions (e.g., to initiate a cleaning cycle, to initiate the operation of the device, to instruct the device timer to begin timing between cleaning cycles, etc.).

In some embodiments, a database engine 704 is configured to facilitate the storage, management, and retrieval of data to and from one or more storage mediums, such as the one or more internal databases described herein including all machine learning. In some embodiments, the database engine 704 is coupled to an external storage system. In some embodiments, the database engine 704 is configured to apply changes to one or more databases. In some embodiments, the database engine 704 comprises a search engine component for searching through thousands of data sources stored in different locations.

In some embodiments, the database engine 704 is operable to collect and store data related to the user's hygiene regimen. The collected data may be used to analyze the user's hygiene regimen, analyze the efficacy of the electronic toothbrush, analyze data received from third-party data sources (i.e., new research related to dental hygiene) and the like along with the programs and frame sets for reiterating new training based upon learned events and future care for either prognosis, practice, exercise or proactive care plan for patient regime and patient engagement.

In some embodiments, the device module 710 is in operable communication with the electronic toothbrush, the protective case, and/or the base to transmit operations instructions. The device module 710 may be in communication with the electronic toothbrush to transmit commands to operate the various electronic components of the electronic toothbrush. For example, the device module 710 may be used to operate the UV lights to initiate a sterilization sequence via the application program and user interface thereof.

In some embodiments, the device module 710 may be operable to remotely control the locking mechanism of the protective case.

In some embodiments, the locking mechanism is a magnetic locking mechanism wherein magnets or mechanical rods, servos, pullies and or gears are bias to close the protective case and prevent the protective case from opening.

In some embodiments, the user module 712 facilitates the creation of a user account for the application system. The user module 712 may allow the user to input various settings, preferences, and personal information to generate the user account. The user account may be used to view and interact with usage data, such as data collected by the application database.

In some embodiments, the scheduling module 714 may be in communication with third-party computing devices and third-party platforms, via the network, to schedule appointments with a dental hygienist, dentist, orthodontist, or other healthcare provider(s).

In some embodiments, the scheduling module 714 may be used to schedule events related to the user's hygiene regimen. For example, the user may schedule a first and second daily cleaning (e.g., at 7 AM and at 9 PM) which schedules an alert and/or notification to be transmitted to remind the user to clean their teeth. The scheduling module 714 may be used to schedule and initiate, via the device module 710, a cleaning cycle of the toothbrush. using the UV lights or other cleaning mechanism.

In some embodiments, the display module 716 is configured to display one or more graphic user interfaces, including, e.g., one or more user interfaces, one or more consumer interfaces, one or more video presenter interfaces, etc. In some embodiments, the display module 716 is configured to temporarily generate and display various pieces of information in response to one or more commands or operations. The various pieces of information or data generated and displayed may be transiently generated and displayed, and the displayed content in the display module 716 may be refreshed and replaced with different content upon the receipt of different commands or operations in some embodiments. In such embodiments, the various pieces of information generated and displayed in a display module 716 may not be persistently stored.

In some embodiments, the display module 716 provides an application program interface and user interface to allow the user to interact with the various functionalities of the system. In such, the display module 716 allows the user to virtually interact, using the user computing device, with the various functionalities described herein.

In some embodiments, the biometric module 718 is in communication with one or more biometric sensors which aid in the identification and verification of a user utilize the electronic toothbrush system. The biometric module 718 may utilize various biometric identification and verification processes and techniques including fingerprint scanning and analysis, facial recognition and analysis, and the like.

In some embodiments, the sensor module 720 is in communication with one or more sensors positioned on the electronic toothbrush, the base, and/or the protective case. The sensors may include one or more motion sensor(s) (e.g., a motion sensor gimbal, a kinetic motion sensor, a pressure sensor, etc.). The sensor module 720 may be operable to transmit alerts or notifications (via the communication module 702).

FIG. 8 illustrates a block diagram of the computer system 600 and databases, including a user database 800, a third-party database 810, and a device database 820. The user database 800 is operable to store user information including the user's personal information, user account information, user hygiene information (i.e., dental history), and a hygiene regimen which is associated with the user profile. The hygiene regimen information may include scheduled cleaning times, cleaning frequency, and cleaning products used (e.g., the type of toothbrush used, the type of toothpaste used, etc.). The third-party database 810 stores information associated with third-parties (e.g., healthcare providers, dentists, dental hygienists, orthodontists, etc.). The device database 820 may be operable to store information associated with the electronic toothbrush system (e.g., information associated with the electronic toothbrush, the base, the protective case, etc.).

FIG. 9 illustrates a block diagram of the electronic toothbrush system 900 which includes a sensor array 902, speakers/microphones 906, UV lights 910, locking mechanism 912, indicators 914, display 916, microprocessor 920, controller 922, biometric sensor 924, and transmitter 926. The electronic toothbrush system 900 is in communication, via network 690, with the computer system 600. The sensor array 902 may include one or more of: one or more motion sensor gimbal(s), one or more kinetic motion sensor(s), one or more accelerometer(s), or other motion sensors known in the arts. The sensor array 902 may also include one or more biometric sensors 924 which are capable of detecting and analyzing biometrics of the user to identify and verify the user's identity.

In some embodiments, the speakers/microphones 906 are provided on the base, the toothbrush, and/or the protective case. The speakers/microphones 906 are operable to emit an audible recording. The audible recording may be used to remind the user to brush their teeth, instruct the user of a status of a dental hygiene regimen status (i.e., to brush for an additional period of time), or to otherwise emit an audible sound which the user can hear.

In some embodiments, the UV lights 910 may be positioned on the toothbrush, on the base, and/or on the protective case. The UV lights 910 are a component of a sterilization process, wherein the UV lights 910 sterilizes the toothbrush components (e.g., the bristle cluster or the head of the toothbrush). The UV lights 910 may in communication with the computer system 600 to allow the user to remotely control the operation of the UV lights 906 via the application program.

In some embodiments, the locking mechanism 912 is a component of the protective case which locks the access door. The locking mechanism 912 may be remotely controlled using the device module which is in communication with the application program.

In some embodiments, the indicators 914 may include a plurality of lights positioned on the electronic toothbrush, the base, and/or the protective case. The indicators may be one or more LED lights capable of displaying one or more colors each indicating a status, an operational function, etc.

In some embodiments, the display 916 may be provided on the base, the protective case, and/or the electronic toothbrush. The display 216 may include a user interface to allow the user to input operational instructions to the electronic toothbrush system. The display 916 may also display a plurality of information associated with the electronic toothbrush system.

In some embodiments, the display 916 include an LCD display, 3-dimensional (3D) display, chat interfaces, and other forms of displays projected as a hologram which can be used to transmit information to the user. The display may be operable to visually display or otherwise visually represent various metrics including the performance of the device, the protective case, base, and other components of the system including the app. The display may include a graphical user interface (GUI) which provides indicators and alerts as well as 2D and/or 3D images and/or videos of the oral cavity, brushing areas, and of the device itself in some instances. The display is in communication with the sensor array to monitor user adherence and display metrics related to the user's adherence to various oral hygiene protocols.

In some embodiments, the display 916 may be used to illustrate x-rays of the patient's oral cavity, 3D representations of the patient's oral cavity and the like. This information may be received from dental records of the patient. This information may be updated over time in a cooperative manner between the patient and the healthcare provider and or system.

In some embodiments, the system utilizes artificial intelligence and machine learning to monitor and manage the adherence to an oral hygiene protocol or care plan. The artificial intelligence and machine learning systems may be operable to suggest and/or enact changes to a protocol based on various data inputs, analysis of the user's performance, current health state, and adherence to their oral hygiene protocol. These systems may be used to further provide feedback to the user based on their performance, adherence, and other health-related metrics which are accessed and/or assessed by the system to improve the prospect of better patient health.

In some embodiments, the display 916 may be externally mounted to the surfaces within the surrounding environment (i.e., a wall, countertop, etc.). Further, the display 916 may be integrated with a mirror.

In some embodiments, the display 916 can be updated in real-time to display various information and metrics which are most relevant at a particular time. This can include real-time updates to the suggested oral hygiene protocol. Information may be received from third party data sources such as the American Dental Association (ADA), from the user's healthcare providers, proctor or bot as provided under any accepted status of protocol etc.

In some embodiments, the display 916 may be in wireless or wired connection to the toothbrush, oral hygiene device(s), the protective case, and/or the base.

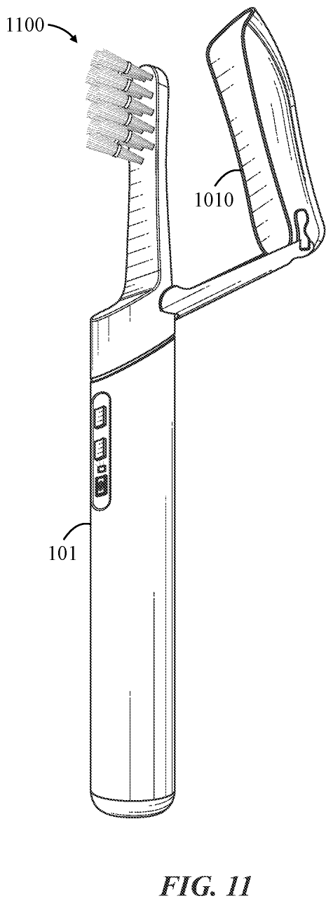

FIG. 10 illustrates the protective case 101 in a closed configuration 1000 and FIG. 11 illustrates the protective case 101 in an open position 1100. As is described herein above, the protective case 101 is designed to be selectively opened or closed using a locking mechanism which is connected to the access door 1010. The locking mechanism is in operable communication with a biometric sensor or other access means which provides access to the toothbrush or other oral hygiene device storage within the protective case 101. This allows the user to open and close the access door 1010 which can provide security and ensure that the proper user is utilizing the toothbrush or oral hygiene device.

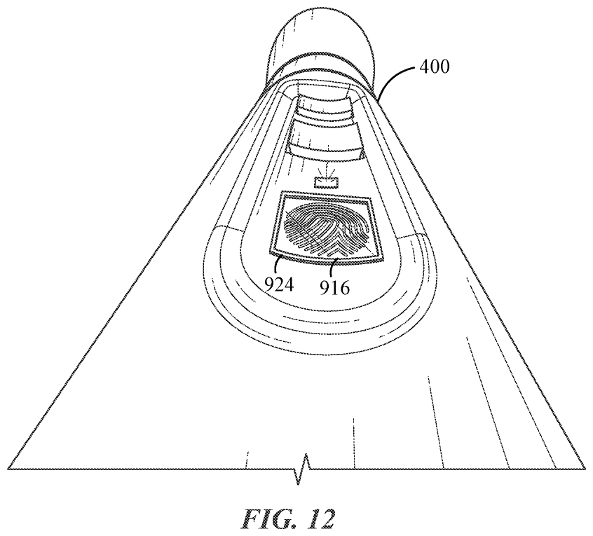

FIG. 12 illustrates a perspective view of the base 400 including the various features described hereinabove. The base 400 may include one or more indicators, one or more displays, a locking mechanism, a charging components, a power storage device, and other components as described herein. The base may also include the biometric sensor 924 which in the displayed example is a fingerprint scanner. However, it is to be noted that various other forms of biometric scanners may be utilized including facial recognition sensors, etc. The biometric sensor 924 may also be a component of the display 916 provided on the base 400.

In some embodiments, the biometric sensor(s) may be used in connection with a smart device camera, the display, external or appliance integrated camera, or other optical recognition devices to scan for user biometrics introduced in any of the appliance form factors described

In some embodiments, the microprocessor 920 is in operable communication with the computing system and includes a controller 922 allowing the user to input operational instructions.

In some embodiments, the biometric sensor 924 is provided on the base, the protective case, and/or the electronic toothbrush. The biometric sensor 924 is capable of analyzing the biometrics of the user, such as by analyzing a fingerprint, analyzing facial biometrics, etc. The biometric sensor 924 may be in operable communication with the biometric module and device module to allow the user to unlock the locking mechanism 912 to gain access to the toothbrush.

In some embodiments, the transmitter 926 is operable to transmit information to a database engine. The transmitter may also transmit information to each module described above which is in communication with the application program.

In some embodiments, the device may include protocols to remain in compliance with various cybersecurity requirements throughout the design stages all the way through to commercialization and use. This may include protocols for ensuring the device is HIMSS and FDA compliant with regards to cybersecurity to the highest degree. This may also include protocols for ensuring health and medical record data is securely stored and transferred. It is also contemplated that the production and development of various aspects of the device conform to healthcare regulations, cybersecurity regulations, and the like.

In some embodiments, the UV light(s) may be positioned about the perimeter of the protective case such that the UV lights shine about the entire perimeter, radius, circumference, etc. of the protective case.

In some embodiments, the UV lights may be programmed to provide a “flash cleanse” of the toothbrush or other oral hygiene device and components thereof which may be activated upon the opening, closing, or other interaction with the toothbrush, oral hygiene device, base, and/or the closing or locking or a protective case or electronic case. The UV light(s) may function to also provide a “flash cleanse” of the protective case, the base, etc.

In some embodiments, the protective case may be used with oral hygiene devices which are not developed as an aspect of the disclosure provided herein. For example, the protective case and/or the base may be utilized with a regular toothbrush to provide the various functions and benefits described herein. This also includes providing the cybersecurity and other security features (i.e., the biometric access) which is described by the present embodiments.

In some embodiments, the bristle cluster and/or the head of the toothbrush is replaceable, disposable, and/or biodegradable. In such, the user may replace the head and/or bristle cluster of the toothbrush as is needed or recommended. It is known and recommended that a toothbrush should be replaced and ordered every 90 days (per ADA recommendations). The system provided herein may include pre-programmed reminders which instruct the user to replace the toothbrush at this time interval (especially in the case where a manual toothbrush is used in conjunction with the smart base and/or protective case described herein). If the user is using the smart toothbrush which is disclosed in the present embodiments, the system may instruct the user to replace the head and/or bristle cluster of the smart toothbrush. The suggestion for replacement is based on the known principle of germ buildup on the toothbrush, bristle cluster, head, protective case, base, etc.

In some embodiments, the components of the system may be constructed of specialized plastics or other materials which are known to be biodegradable, organic, or otherwise easily disposed of to cause a “zero carbon footprint” to the environment.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. An electronic toothbrush system, comprising:
a toothbrush including a handle portion and a head portion, the head portion including a bristle cluster;
a sensor array in communication with a housing of the toothbrush, the sensor array to detect the usage of the toothbrush;
a microprocessor in electrical communication with the sensor array;
one or more I/O devices in electrical communication with the microprocessor, the I/O devices to provide an alert, a sound, or an indication;
a protective case dimensioned to at least partially receive the toothbrush and to encase the head portion; and
a locking mechanism to prevent unwanted or unpermitted access to the toothbrush.

2. The electronic toothbrush system of claim 1, wherein the sensor array includes a biometric sensor capable of scanning a biometric element of a human.

3. The electronic toothbrush system of claim 1, wherein toothbrush is mountable to a base to secure the toothbrush while the toothbrush is inoperative, wherein the base includes a second sensor array including a at least one motion sensor and second biometrics sensor, wherein the base includes a second set of at least one I/O devices to provide a sound, an alert, or an indicator, and wherein the base includes a second microprocessor in operable communication with the second sensor array, the second set of at least one I/O devices, and the second biometrics sensor.

4. An electronic toothbrush system, comprising:
a toothbrush including a handle portion and a head portion, the head portion including a bristle cluster;
a sensor array in communication with a housing of the toothbrush, the sensor array to detect a usage of the toothbrush and to detect a presence of a human and an identity of the human using a biometric sensor;
a microprocessor in electrical communication with the sensor array;
one or more I/O devices in electrical communication with the microprocessor, the I/O devices to provide an alert, a sound, or an indication;
a protective case dimensioned to at least partially receive the toothbrush and to encase the head portion;
one or more UV lights to provide an antiseptic environment within the protective case; and
a base to provide a secure mount for the toothbrush.

5. The electronic toothbrush system of claim 4, wherein the toothbrush is in operable communication with the base and the protective case via a wireless communication protocol.

6. The electronic toothbrush of claim 5, wherein the toothbrush includes a power supply to power the microprocessor and a motor enclosed within the housing of the toothbrush, the motor to provide multi-directional movement of the bristle cluster.

7. The electronic toothbrush of claim 6, wherein the microprocessor is in operable communication with a memory to store operational instructions associated with an oral hygiene regimen.

8. The electronic toothbrush of claim 7, wherein the one or more I/O devices are in operable communication with the microprocessor to emit a sound or an indicator associated with the oral hygiene regimen.

9. The electronic toothbrush system of claim 8, further comprising a locking mechanism to selectively secure the protective case in a closed position.

10. The electronic toothbrush system of claim 9, wherein toothbrush is mountable to a base to secure the toothbrush while the toothbrush is inoperative, wherein the base includes a second sensor array including a at least one motion sensor and second biometrics sensor, wherein the base includes a second set of at least one I/O devices to provide a sound, an alert, or an indicator, and wherein the base includes a second microprocessor in operable communication with the second sensor array, the second set of at least one I/O devices, and the second biometrics sensor.

11. The electronic toothbrush system of claim 10, wherein the protective case is a travel case including a locking mechanism to secure the toothbrush within the travel case.

12. An electronic toothbrush system, comprising:
a toothbrush including a handle portion and a head portion, the head portion including a bristle cluster;
a sensor array in communication with a housing of the toothbrush, the sensor array to detect a usage of the toothbrush and to detect a presence of a human and an identity of the human using a biometric sensor;
a microprocessor in electrical communication with the sensor array;
one or more I/O devices in electrical communication with the microprocessor, the I/O devices to provide an alert, a sound, or an indication;
a protective case dimensioned to at least partially receive the toothbrush and to encase the head portion;
one or more UV lights to provide an antiseptic environment within the protective case; and
a computer system in operable communication with an application program operated by a user computing device, the application program in communication, via a network or near frequency communication protocol, to the toothbrush to execute computer-readable instructions to establish operational protocols to aid in the formation of a hygiene regimen carried out by the utilization of the toothbrush.
